(19) United States
(12) Patent Application Publication
(10) Pub. No.: US 2009/0157012 A1
(43) Pub. Date: Jun. 18, 2009

(54) Syringe with extendable and retractable needle

(75) Inventor: Luc Magne, Victoria British Columbai (CA)

Correspondence Address: KLARQUIST SPARKMAN, LLP, 121 SW SALMON STREET, SUITE 1600, PORTLAND, OR, 97204, US

(73) Assignee: University of Victoria Innovation and Development Corporation

(21) Appl. No.: 12/002,117

(22) Filed: Dec. 13, 2007

(51) Int. Cl. A61M 5/32; A61M 5/00

(52) U.S. Cl. 604/198; 604/110; 604/177

(57) ABSTRACT

A syringe for use with a carpule and needle is provided. The syringe comprises: (i) a holder for accepting the carpule, the holder comprising at least two linearly disposed first mating members; (ii) a plunger for engagement with the holder; and (iii) a shell for slidably housing the holder, the shell having a distal aperture and a proximal aperture, with a bore therebetween, the shell comprising at least one integral biased member proximate to the proximal aperture, the biased member having a second mating member proximate to a distal end, for mating with the first mating member, such that in a first position, the needle is housed within the shell and in a second position, the needle extends at least partially from the shell.

### FIELD

The technology is a syringe for use with a carpule and needle, more specifically, a retractable syringe that permits the needle to be obscured from view as desired.

### BACKGROUND

In recent years there has been concern over the safety of syringes. Conventional needles are provided with a cap or cover which is removed after the needle is affixed to the syringe. At this time, there is a potential for the practitioner or the patient to be inadvertently stuck with the needle. For this reason, syringes with retractable needles have been developed that permit the practitioner to expose the needle only when necessary.

One such device is disclosed in U.S. Pat. No. 6,656,164. A housing has a plunger and a carpule housing. A spacer is provided between a lead screw and the carpule housing allowing the latter (in combination with plunger) to extend and retract with respect to the former. The carpule housing comprises an internally threaded portion for engagement with threaded outer surface of a second carpule housing. On the interior surface of the second housing are located a first pair of locking lugs. Disposed distally of the housing is a safety shield. The proximal end of the safety shield comprises a second pair of locking lugs. The first pair of locking lugs and the second pair of locking lugs are designed in a conventional manner to achieve reversible engagement the housing and the safety shield.

When it is desired to inject a material, a forward switch in the housing is actuated. This drives a motor which results in extension of the lead screw and plunger. The plunger then abuts rubber stopper. The respective dimensions of the carpule housing and safety shield are selected so that the linear force necessary to overcome friction between the two elements is less than the force required to move rubber stopper—i.e., avoiding premature dispensing of the biocompatible material. This results in movement of the entire carpule/carpule housing combination within a safety shield until the head abuts against the tapered portion near the distal end of the interior of the safety shield. At this point, the needle is extended from safety shield and continued application of linear force by the plunger results in movement of rubber stopper thereby injecting biocompatible material from the now exposed needle.

A simplified syringe that provides a different needle positions is required. It is an object of the present technology to overcome the deficiencies in the prior art.

### SUMMARY

The present technology is a syringe that provides for a number of different needle positions in the absence of a complex mechanism for locating the carpule within a case. Specifically, the present technology is a syringe for use with a carpule and needle, comprising:

(i) a holder for accepting the carpule, the holder comprising at least two linearly disposed first mating members;

(ii) a plunger for engagement with the holder; and

(iii) a shell for slidably housing the holder, the shell having a distal aperture and a proximal aperture, with a bore therebetween, the shell comprising at least one integral biased member proximate to the proximal aperture, the biased member having a second mating member proximate to a distal end, for mating with the first mating member, such that in a first position, the needle is housed within the shell and in a second position, the needle extends from the shell.

In one aspect, the first mating member and the second mating member are a depression-protrusion pair.

In another aspect the first mating member is a depression and the second mating member is a protrusion.

In another aspect the proximal aperture and the holder are suitably shaped for slidable engagement with one another in one of two selected orientations.

In another aspect the holder further comprises a channel in gaseous communication with the ambient environment, to permit self aspiration of the holder.

In another aspect at least a portion of the shell adjacent the carpule comprises a window.

In another aspect the window is comprised of transparent material.

In another aspect the shell is transparent.

In another aspect the shell is textured proximate to the distal aperture, to conceal the needle.

In another aspect the shell proximate to the proximal aperture has an outer coating of a compliant polymeric material.

In another aspect the shell has two integral biased members.

In another aspect the compliant polymeric material is shaped into a child-friendly shape.

In another embodiment, a combination is provided. The combination comprises:

(a) a needle;

(b) a carpule; and

(c) a syringe, the syringe comprising:
(i) a holder for accepting the carpule, the holder comprising at least two linearly disposed first mating members;
(ii) a plunger for engagement with the holder; and
(iii) a shell for slidably housing the holder, the shell having a distal aperture and a proximal aperture, with a bore therebetween, the shell comprising at least one integral biased member proximate to the proximal aperture, the biased member having a second mating member proximate to a distal end, for mating with the first mating member, such that in a first position, the needle is housed within the shell and in a second position, the needle extends at least partially from the shell.

In one aspect of the combination, the first mating member and the second mating member are a depression-protrusion pair.

In another aspect of the combination, the first mating member is a depression and the second mating member is a protrusion.

In another aspect of the combination, the proximal aperture and the holder are suitably shaped for slidable engagement with one another in one selected orientation.

In another aspect of the combination, the holder further comprises a channel in gaseous communication with the ambient environment, to permit self aspiration of the holder.

In another aspect of the combination, at least a portion of the shell adjacent the carpule comprises a window.

In another aspect of the combination, the window is comprised of transparent material.

In another aspect of the combination, the shell is transparent.

In another aspect of the combination, the shell is textured proximate to the distal aperture, to conceal the needle.

In another aspect of the combination, the shell proximate to the proximal aperture has an outer coating of a compliant polymeric material.

In another aspect of the combination, the shell has two integral biased members.

In another aspect of the combination, the compliant polymeric material is shaped into a child-friendly shape.

In another aspect of the combination, the shell is injection molded.

In another aspect of the syringe, the shell is injection molded.

The foregoing and other features and advantages of the invention will become more apparent from the following detailed description, which proceeds with reference to the accompanying figures.

### BRIEF DESCRIPTION OF THE DRAWINGS

FIG. 1 is a perspective view of the technology.

FIG. 2 is a perspective view of the holder of the technology of FIG. 1.

FIG. 3 are longitudinal sections showing the depressions in the holder and the biasing members of the shell.

FIG. 4 are longitudinal sections showing the inside view and the outside view of the shell of the technology of FIG. 1.

FIG. 5 is a perspective view of the technology with the needle retracted.

### DETAILED DESCRIPTION

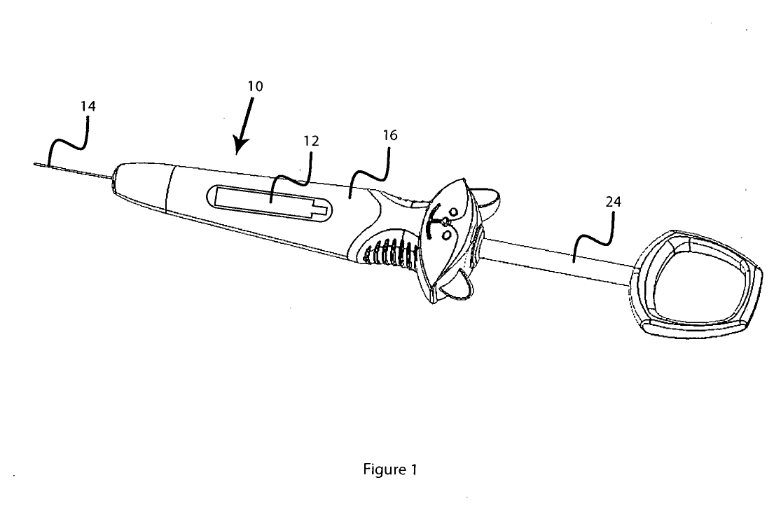

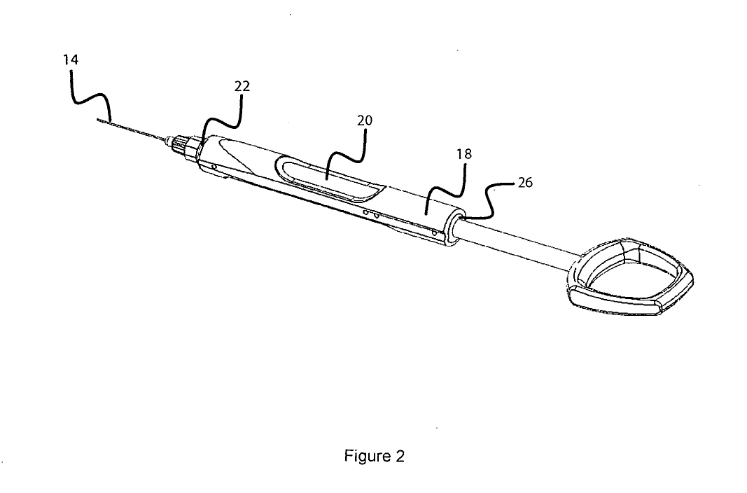

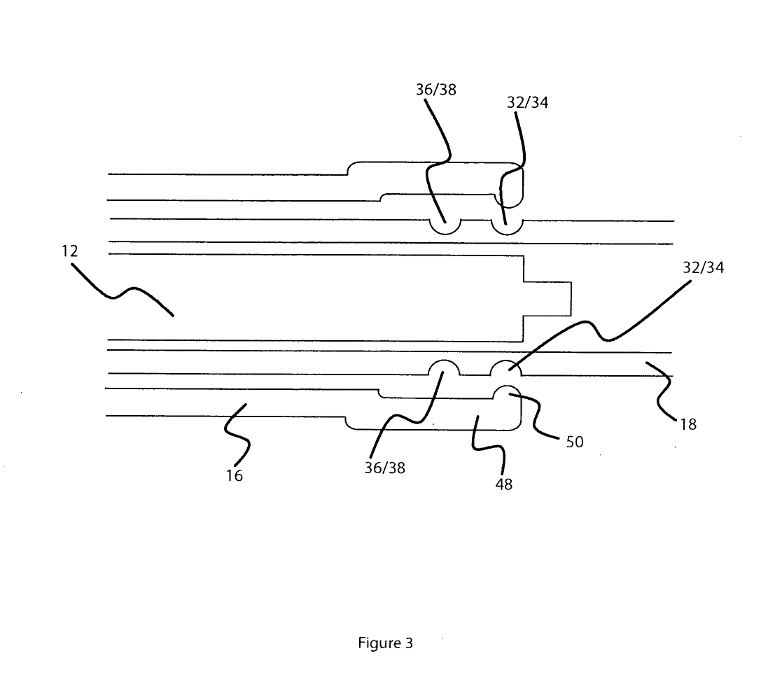

A syringe, generally referred to as 10 is shown in FIG. 1. The syringe 10 is for use with a carpule 12 and a needle 14. An outer shell 16 covers a generally cylindrical holder 18 (see FIG. 2). The holder 18 holds the carpule 12. As shown in FIG. 2 the holder 18 has a window 20 that permits viewing of the carpule 12. A distal end 22 of the holder 18 accepts standard needles 14. A proximal end 26 is shaped to mate with the inner wall 28 of the outer shell 16, which defines a generally oval bore 30 (see FIG. 3). The holder 18, therefore can be accepted by the shell 16 in one or the other of two opposing orientations. This ensures visibility of the carpule 12 to the user. A plunger 24 extends from the proximal end 26.

The shell 16 is in slidable engagement with the holder 18. As shown in FIG. 3, the holder 18 has two sets of depressions located on opposing sides of the holder 18. A first depression 32 defines a first position 34 that is proximate to the proximal end 26 and a second depression 36 defines a second position 38, located distal to the first depression 32. The distance between the first depression 32 and the second depression 36, and indeed, any additional depressions, determines the relationship of the needle 14 to the shell 16.

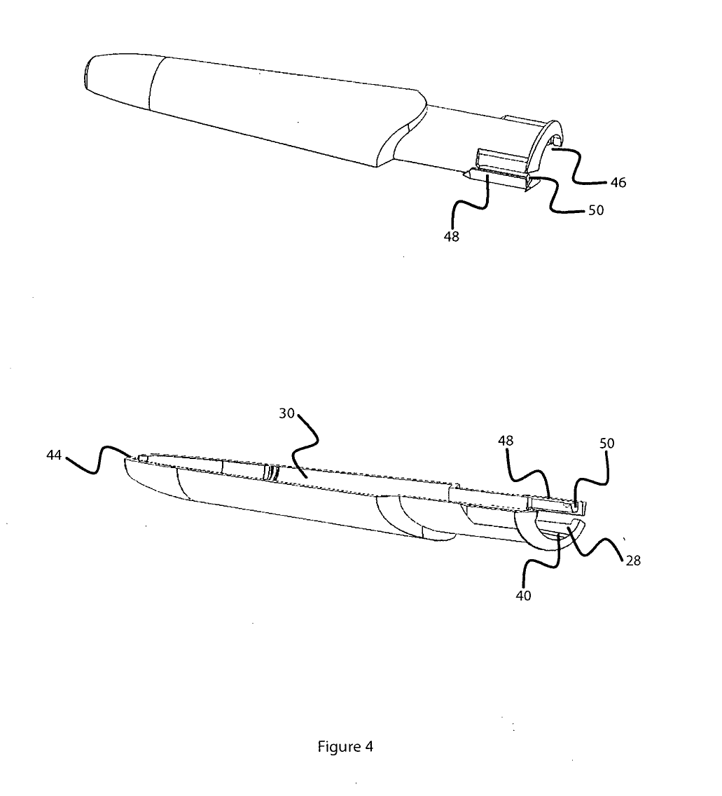

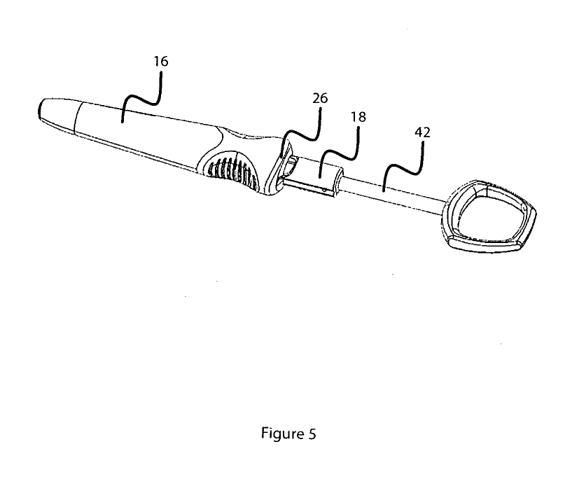

As shown in FIG. 4, the shell 16 has a distal aperture 44, a proximal aperture 46 and a bore 30 therebetween. Two biasing members 48 are located on opposing sides of the inner wall 28, proximate to the proximal aperture 46. The biasing members 48 are integral with the shell 16. Each biasing member 48 has a protrusion 50 proximate to the biasing member distal end 52 for mating with the depressions 32, 36. In the first position 34 (corresponding to the first depression 32), the needle 14 is retracted within the shell 16 (FIG. 5), in the second position 38 (corresponding to the second depression 36) the needle 14 is exposed (FIG. 1).

The holder 18 has a channel 40 extending from the proximal end 26 to the carpule 12, to allow for aspiration. The shell 16 is textured proximate to the distal aperture 44, to conceal the needle 14. Proximate to the proximal aperture 46, the shell 16 is coated or otherwise covered with a polymeric material that is similar to that found on toothbrushes.

The shell can be injection molded in two identical pieces that are then joined together. Alternatively, a single mold can be used.

In use, the needle is initially in the retracted position, as shown in FIG. 5. The plunger 24 is extended, and the protrusion 50 is mated with the first depression 32. The user then applies pressure to the plunger 24, the protrusion 50 pops out of the first depression 32, and the holder 18 slides into the shell 16 until the second depression 36 is reached. Further pressure can again force the protrusion 50 to pop from the second depression 36 and move to subsequent depressions, each time, advancing the needle outward from the shell 16, as shown in FIG. 1. Upon reaching the final depression, further force will cause the plunger 24 to urge the contents of the carpule 12 from the carpule 12, through the needle 14.

The foregoing is an embodiment of the technology. As would be known to one skilled in the art, variations that do not alter the scope of the invention are contemplated. For example, but not limited to, a variety of mating members could be used—the location of the depression and the protrusion could be reversed, interdigitating members could be employed, for example. The entire shell can be transparent, or portions of it can be transparent. Further, a window may be present in the shell, which can be filled with a transparent material or left as an opening. The textured portion of the shell could be coloured or otherwise treated to obscure the needle. The plunger can be made of a clear polymer. The polymeric material can be molded into a patient friendly shape, such as an animal head, an animal, or a tooth.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. I therefore claim as my invention all that comes within the scope and spirit of these claims.

I claim:

1. A syringe for use with a carpule and needle, the syringe comprising:
(i) a holder for accepting the carpule, the holder comprising at least two linearly disposed first mating members;
(ii) a plunger for engagement with the holder; and
(iii) a shell for slidably housing the holder, the shell having a distal aperture and a proximal aperture, with a bore therebetween, the shell comprising at least one integral biased member proximate to the proximal aperture, the biased member having a second mating member proximate to a distal end, for mating with the first mating member, such that in a first position, the needle is housed within the shell and in a second position, the needle at least partially extends from the shell.

2. The syringe of claim 1 wherein the first mating member and the second mating member are a depression-protrusion pair.

3. The syringe of claim 2 wherein the first mating member is a depression and the second mating member is a protrusion.

4. The syringe of claim 3 wherein the proximal aperture and the holder are suitably shaped for slidable engagement with one another in one of two selected orientations.

5. The syringe of claim 4, wherein the holder further comprises a channel in gaseous communication with the ambient environment, to permit self aspiration of the holder.

6. The syringe of claim 5 wherein at least a portion of the shell adjacent the carpule comprises a window.

7. The syringe of claim 6 wherein the window is comprised of transparent material.

8. The syringe of claim 7 wherein the shell is transparent.

9. The syringe of claim 8 wherein the shell is textured proximate to the distal aperture, to conceal the needle.

10. The syringe of claim 9 wherein the shell proximate to the proximal aperture has an outer coating of a compliant polymeric material.

11. The syringe of claim 10 wherein the shell has two integral biased members.

12. The syringe of claim 11 wherein the compliant polymeric material is shaped into a child-friendly shape.

13. A combination comprising:
(a) a needle;
(b) a carpule; and
(c) a syringe, the syringe comprising:
(i) a holder for accepting the carpule, the holder comprising at least two linearly disposed first mating members;
(ii) a plunger for engagement with the holder; and
(iii) a shell for slidably housing the holder, the shell having a distal aperture and a proximal aperture, with a bore therebetween, the shell comprising at least one integral biased member proximate to the proximal aperture, the biased member having a second mating member proximate to a distal end, for mating with the first mating member, such that in a first position, the needle is housed within the shell and in a second position, the needle extends at least partially from the shell.

14. The syringe of claim 13 wherein the first mating member and the second mating member are a depression-protrusion pair.

15. The syringe of claim 14 wherein the first mating member is a depression and the second mating member is a protrusion.

16. The combination of claim 15 wherein the proximal aperture and the holder are suitably shaped for slidable engagement with one another in one selected orientation.

17. The combination of claim 16, wherein the holder further comprises a channel in gaseous communication with the ambient environment, to permit self aspiration of the holder.

18. The combination of claim 17 wherein at least a portion of the shell adjacent the carpule comprises a window.

19. The combination of claim 18 wherein the window is comprised of transparent material.

20. The combination of claim 17 wherein the shell is transparent.

21. The combination of claim 20 wherein the shell is textured proximate to the distal aperture, to conceal the needle.

22. The combination of claim 21 wherein the shell proximate to the proximal aperture has an outer coating of a compliant polymeric material.

23. The syringe of claim 22 wherein the shell has two integral biased members.

24. The combination of claim 23 wherein the compliant polymeric material is shaped into a child-friendly shape.

25. The syringe of claim 11 wherein the shell is injection molded.
